(19) United States
(12) Patent Application Publication
(10) Pub. No.: US 2023/0011654 A1
(43) Pub. Date: Jan. 12, 2023

(54) CATHETER COMPRISING A SPRAYING DEVICE AND A CONTROL UNIT

(71) Applicants: UNIVERSITE DE BORDEAUX, BORDEAUX (FR); CENTRE HOSPITALIER UNIVERSITAIRE DE BORDEAUX, TALENCE CEDEX (FR)

(72) Inventors: Eric DUMAS DE LA ROQUE, BORDEAUX (FR); Michael FAYON, PESSAC (FR)

(21) Appl. No.: 17/782,011

(22) PCT Filed: Dec. 18, 2020
(86) PCT No.: PCT/EP2020/087061

§ 371 Date: Jun. 2, 2022

(30) Foreign Application Priority Data
Dec. 18, 2019 (FR) FR1914751

(51) Int. Cl. A61B 1/00

(52) U.S. Cl. CPC A61B 1/00045; A61B 1/0008; A61B 1/00165; A61B 1/00154

(57) ABSTRACT

A catheter for delivering a medicinal liquid or a powder in spray form into the trachea of a subject, includes a deformable body including a spray device for expelling a liquid or a powder introduced into the catheter as a spray, the body of the catheter being configured to generate an image of a zone in a longitudinal direction of the body.

### FIELD OF THE INVENTION

The invention relates to a catheter and a system for administering microdroplets into the trachea of a subject, notably a premature subject.

### PRIOR ART

Today, one birth out of eight in the United States and around one out of fourteen in Europe is premature. Around 20% of these premature babies suffer from respiratory distress syndrome, known by the acronym RDS. RDS is capable of causing the death of the baby if the latter is not cared for immediately, notably within the hour following its birth.

Indeed, the earlier the baby is born (before the gestation period of 37 weeks), the less its body is ready to face the outside world. It is at the third quarter of pregnancy when pulmonary capacity increases rapidly. In parallel, the refinement of the alveolar walls of the lungs enables an increase in the surface area for gaseous exchanges. Any early interruption of this maturation process can lead to an alteration of the respiratory function and the pulmonary physiology.

The caring of premature babies suffering from RDS generally requires the administration of a surfactant and an initial oxygenation. However, the administration of a liquid surfactant into the respiratory tracts with existing methods comprises several drawbacks.

A first drawback is the quantity of liquid surfactant administered into the pulmonary tracts which causes a drowning effect for the baby. Indeed, the volume administered to a premature baby is equivalent for an adult to a volume of around 200 mL or to 2.5 mL per kilogram of the subject.

A second drawback is that the method used is invasive.

It is in fact necessary to approach the entrance of the larynx using a laryngoscope. The entrance of the larynx is very narrow, particularly in premature subjects. This procedure is tricky and difficult to carry out, even for an experienced practitioner. Furthermore, this intrusion is also painful for the infant. It thus often involves analgesia and/or sedation to reduce the pain of the infant and to facilitate the procedure. The problem is then posed of the administration of an analgesic and/or sedative, which may be very tricky for premature babies.

A third drawback is the tolerance of the child during the administration of the surfactant which is generally administered in liquid form at the level of the trachea, generating a drowning effect in the child. The surfactant can cause complications such as discomfort through a decrease in oxygenation or a slowing down of the heart rhythm. The administration of the liquid must thus be prudent and often discontinuous in the event of discomfort of the baby.

To try to overcome these drawbacks, the patent document WO2015/059037 is known describing a system for administering a medicine comprising a pulmonary surfactant comprising a catheter comprising a first channel suited for conveying into the pharyngeal region of the subject a flow of medicinal liquid and a second channel for conveying a flow of pressurized gas. A connection between the first and second channels enables the nebulization of the medicinal liquid at the junction between the liquid and the pressurized gas.

However, this type of device always requires the use of a laryngoscope to guide the catheter, involving all the aforementioned drawbacks.

In addition, this type of catheter requires two channels to bring a flow of gas and the medicinal liquid to the distal end of the catheter. The catheter must thus be very wide and may not be suited for its introduction beyond the vocal chords on premature subjects.

Finally, this type of catheter allows the administration of a nebulized medicinal liquid into the pharyngeal region, this very upstream region allows a part of the surfactant to pass through the esophagus, which reduces the efficiency of the medicine and imposes increasing the volume of liquid to inject and the injection time often exceeding 1 minute, or even 10 to 15 minutes.

Another drawback of this type of catheter results from the fact that part of the medicinal liquid is blocked at the level of the vocal chords of the infant, thus preventing the medicinal liquid from reaching the lungs.

The present invention aims to provide a catheter and a system for administering a medicinal liquid while being free of the cited drawbacks of the prior art.

One objective of the invention is to provide a catheter enabling the release of a medicinal liquid in the form of microdroplets as close as possible to the pulmonary tracts, preferentially in the trachea.

Another objective of the invention is to enable a simplified guiding of the catheter in the larynx of a premature subject without resorting to a laryngoscope or to another invasive method and more rapidly. Further, the invention aims to enable an operation to be carried out on the subject by a single person.

Another objective of the invention is to provide a system enabling the caring of a premature baby suffering from respiratory distress syndrome without resorting to analgesia or to sedation of the premature subject.

Another objective of the invention is to provide a system enabling the treatment of respiratory distress syndrome while minimizing its impact on the parameters of ventilatory support of the patient.

Another objective of the invention is to provide a catheter that is inexpensive and simple to manufacture and use.

### SUMMARY OF THE INVENTION

The invention relates to a catheter for delivering a medicinal liquid or a powder in spray form into the trachea of a subject, characterized in that said catheter comprises a deformable body comprising a spray device for expelling a liquid or a powder introduced into the catheter as a spray, said catheter body comprising, further, an optic configured to generate an image of a zone, said zone being situated in the extension of the distal end of said catheter.

According to an example, the zone is situated in a longitudinal direction of the body from the distal end of the catheter.

The invention also relates to a catheter for delivering a medicinal liquid in the form of microdroplets into the trachea of a subject. The catheter comprises a deformable body in which a moveable element translationally moves beyond the body of the catheter. The moveable element comprises a spray device for expelling a liquid introduced into the catheter in the form of microdroplets. Said catheter body comprises, further, an optic configured to generate an image of the distal end of the moveable element in translated position.

The invention also relates to a catheter for delivering a medicinal liquid or a powder in spray form into the trachea of a subject, such as a premature newborn subject. Said catheter is designed to be inserted into the trachea through the vocal chords of the subject and comprises a deformable body comprising a spray device for expelling a liquid or a powder introduced into the catheter as a spray, said catheter body comprising, further, a lumen for the passage of an optic arranged to generate an image of a zone, said zone being situated in the extension of the distal end of said catheter and comprising a drive member for the folding of the catheter body along a predetermined orientation.

The deformation of the body of the catheter may be, for example, an elastic deformation or instead a deformation ensured by a mechanical link of angular link, pivot link or swivel link type.

According to one embodiment, the body of the catheter comprises a support for maintaining an optic. By extension, the body of the catheter may be understood as a body comprising an optic. The invention also relates to embodiments in which the optic could be removable from the body of the catheter. The invention advantageously makes it possible to deliver a medicinal liquid into the trachea, that is to say beyond the vocal chords when the catheter is introduced through the mouth or by nasal route. The invention also advantageously makes it possible to be free of the use of a laryngoscope. Indeed, the practitioner can easily introduce the end of the body by guiding the distal part and optionally by visualizing the images of the optic. This embodiment thus makes it possible to be free, before administering care, of analgesia or sedation of the premature baby linked to the laryngoscope. The invention makes it possible to provide a catheter being free of the cited drawbacks of the prior art.

In one embodiment, the catheter comprises a first member for driving a translation of the moveable element, the distance of translation of the moveable element being able to be driven over a range of positions comprised between 0.5 cm and 2 cm or between 0.5 cm and 4.5 cm.

This embodiment advantageously makes it possible to treat a premature subject by inserting the moveable element into the trachea through the vocal chords when the catheter body is in the larynx.

In one embodiment, the catheter comprises a second member for driving an orientation of a distal part of the catheter body with a proximal part of said body so as to control said orientation of the distal part in a predetermined direction. The drive member makes it possible to drive, to trigger or to control this orientation.

This embodiment advantageously makes it possible to facilitate the guiding of the distal part of the catheter body in the larynx by adjusting the orientation of the distal part in a controlled manner or remotely. The practitioner can visualize the images of the optic to locate the entrance of the larynx and to trigger the orientation of the catheter head to guide the latter through the entrance of the larynx. The invention thus makes it possible to be free of the drawbacks of the prior art, to accelerate the introduction of the catheter into the larynx, to reduce the trauma of the subject and to be free of the use of a laryngoscope.

In one embodiment, the catheter comprises a positioning piece intended to cooperate with the mouth of a subject, said positioning piece extending radially at least partially around the body of the catheter to form a buccal support limiting (or designed to limit) the volume of air transiting outside the catheter between the inside and the outside of the mouth. The buccal support also makes it possible to maintain the catheter in the axis of the pharynx to facilitate the guiding of the catheter.

In one embodiment, the positioning piece is translationally moveable on the body of the catheter. In one embodiment, the positioning piece comprises a guide to orientate the introduction of the catheter into the respiratory tracts of said subject.

The positioning piece acts as a pivotal point. This pivotal point advantageously makes it possible to facilitate the guiding of the catheter body by the practitioner.

Said positioning piece also makes it possible to ensure an at least partial leak tightness to air through the mouth of the subject. The leak tightness of the positioning piece improves the efficiency of the respiratory support system of the premature infant, notably when the respiratory support system is introduced by nasal route.

The translational freedom of the positioning piece advantageously makes it possible to improve the guiding of the body as of the start of the insertion thanks to the pivotal point constituted by said positioning piece in cooperation with the mouth.

Finally, this piece makes it possible to obtain a bearing point of the catheter allowing the operator to free one hand to manipulate the catheter to guide it.

In one embodiment, the catheter comprises a passage channel for a liquid emerging at the end of the spray device.

In one embodiment, the spray device is designed to deliver a medicinal liquid in the form of aerosol or a medicinal powder in the form of aerosol or spray.

In one embodiment, the spray device comprises:
an insert arranged inside the passage channel and extending longitudinally along the passage channel, said insert having an outer surface comprising at least one substantially helicoidal furrow extending from the proximal end up to the distal end of the insert and suited for the passage of the medicinal liquid;
a chamber for receiving said volume of medicinal liquid at the outlet of a furrow of the insert;
a channel pressurizing a volume of medicinal liquid within an orifice arranged in the extension of said chamber.

Such a spray device advantageously makes it possible to produce an aerosol by pressurizing the medicinal liquid.

In one embodiment, the passage channel further comprises a stop limiting the translational movements of the insert. This stop advantageously makes it possible to secure the position of the insert in the passage channel despite the flow of pressurized liquid.

In one embodiment, said optic is arranged at the distal end of the body of the catheter.

The lumen for the passage of an optic (6) is designed to receive an optic fiber extending along said lumen. The optic fiber may extend along said lumen, preferentially up to the distal end of the catheter body. In one embodiment, the optic fiber passage lumen comprises, at the distal end of the catheter body, a transparent and leak tight wall to protect the optic fiber passage lumen from contaminations of the external environment. Preferentially, the wall is a sterile wall. In one embodiment, the optic fiber is also designed to light up a zone situated in the distal extension of the catheter.

In one embodiment, the distal end of the body comprises a rounded shape. This rounded shape advantageously makes it possible to reduce the trauma of the subject during contact between the distal end of the body and the respiratory tracts of the subject.

In one embodiment, the outer diameter of the catheter body intended to be inserted into the respiratory tracts of the subject is comprised between substantially 1 mm and 5 mm or is less than substantially 5 mm, preferentially less than 3 mm. One advantage of this diameter is to be able to insert the catheter if necessary into the respiratory tracts of a premature newborn baby.

The invention also relates to a medical system comprising a catheter according to the invention, and comprising a command unit. The command unit comprises a reservoir for receiving a volume of medicinal liquid, and a device for commanding the administration of the medicinal liquid driving (or for driving) a high pressure pump.

The reservoir comprises means for controlling the temperature of the medicinal liquid or the medicinal powder in the reservoir.

In one embodiment, the medical system comprises a device for detecting and/or measuring the respiratory cycle of the subject, the administration of the medicinal liquid by the command device being synchronized with the respiratory cycle of the subject. The administration of the medicinal liquid may advantageously be carried out during the inspiratory phase of the subject.

This synchronization advantageously makes it possible to favor the transport of the medicinal liquid in the form of microdroplets to the alveoli of the lung thanks to the respiration of the subject and to decrease its expulsion by the respiratory tracts out of the trachea.

In one embodiment, the medical system further comprises a ventilation device for the respiratory assistance of the subject. The medical system may comprise a display screen to display images of the optic.

In one embodiment, the command unit comprises a means for actuating the second member for driving the catheter.

The invention also relates to an intubation system comprising a catheter according to the invention and comprises an intubation probe. The catheter is then designed to be inserted at least in part into the intubation probe. Said system advantageously allows the practitioner, during the use of the catheter, to introduce rapidly an intubation probe by sliding the intubation probe along the catheter. The intubation probe is then, in the event of serious discomfort of the subject, much faster to insert up to the larynx than with a laryngoscope and enables a securement of the subject in cardiorespiratory terms.

### BRIEF DESCRIPTION OF THE FIGURES

Other characteristics and advantages of the invention will become clear on reading the detailed description that follows, with reference to the appended figures, which illustrate:

FIG. 1A: a transparency view of a catheter body according to a first embodiment of the invention.

FIG. 1B: a sectional view of the catheter body wherein the passage channel comprises a concentric portion in retracted position.

FIG. 2A: a perspective view of a catheter body according to the first embodiment wherein the passage channel is translated in the distal direction beyond the catheter body.

FIG. 2B: a sectional view of the catheter body wherein the passage channel comprises a concentric portion in translated position beyond the catheter body.

FIG. 3: a sectional view of the section of the body of the catheter according to a first embodiment of the invention.

FIG. 4: a sectional view of the section of the catheter body according to a second embodiment wherein the body comprises an activating wire and a ventilation lumen.

FIG. 5: a perspective view of a medical system comprising a catheter according to one embodiment of the invention wherein the catheter body comprises an orientable portion and wherein the system comprises a display means.

FIG. 6: a sectional view of the moveable element of the passage channel according to one embodiment making it possible to deliver the medicinal liquid in the form of an aerosol.

FIG. 7: a diagram of a subject into whom is introduced the distal part of the catheter body.

FIG. 8: a diagram of a subject into whom the catheter rod is introduced up to the larynx.

FIG. 9: an enlarged view in which the distal end of the catheter body is in the larynx facing the vocal chords.

FIG. 10: an enlarged view in which the distal end of the catheter rod is in the larynx facing the vocal chords and the moveable element is introduced into the trachea through the vocal chords.

FIG. 11: an enlarged view in which the medicinal liquid is sprayed into the trachea in the form of microdroplets.

FIG. 12: an enlarged view in which the distal end of the catheter is inserted into the trachea through the vocal chords.

FIG. 13: a sectional view of the section of the body of the catheter according to another embodiment of the invention.

### DETAILED DESCRIPTION

In the remainder of the description, the following terms should be understood in light of their definition hereafter:

“Distal” is taken to mean the side the farthest from a point of the catheter held by the practitioner during the use thereof.

“Proximal” is taken to mean the side the nearest to a point of the catheter held by the practitioner during the use thereof.

“Channel” is taken to mean an artificial conduit making it possible to convey a liquid.

“Lumen” is taken to mean an orifice extending along the longitudinal axis of the body of the catheter.

“Microdroplets” or “microdrops” is taken to mean drops of liquid separated by air or a gas, the diameter of these drops being comprised between 1000 μm and 5 μm.

“Spray” is taken to mean a liquid or solid substance broken down into very fine particles. When it is a liquid substance, the spray leads to the generation of microdroplets. When it is a solid substance, the spray leads to the generation of particles of material, for example in the form of a powder.

In one embodiment, the “spray” may comprise a nebulization or an aerosolization.

“Nebulization” is taken to mean the spray of microdroplets by the encounter of a liquid and a pressurized gas.

“Aerosolization” is taken to mean the spray of microdroplets produced when a pressurized liquid passes through a conduit of which the outlet comprises a shape particularly favorable to the creation of microdrops.

The invention pertains to a catheter 1 comprising a catheter body 2 for administering a medicinal liquid, notably into the trachea of a premature subject. The invention makes it possible to treat respiratory distress syndrome secondary to pulmonary immaturity requiring the administration of surfactant, but also all pulmonary pathologies requiring the administration of a medicinal liquid, into the lung tracts. Among these other pulmonary pathologies, pathologies such as pulmonary hemorrhage, pneumonias, broncho-pulmonary dysplasia and prolonged respiratory insufficiency in patients ventilated in a chronic manner may be noted. The invention also makes it possible to treat newborn babies presenting a secondary deterioration of the surfactant or a defect of its recycling.

In one embodiment, the medicinal liquid may be replaced by a medicinal powder. The spray is then a spray of a medicinal powder.

The invention also pertains to a medical system 300 comprising said catheter 1 according to the invention.

### Catheter Rod

The proximal part of the body 2 may be connected to a command center 100, also called command unit. The command center 100 may comprise a user interface. According to different embodiments, the command center may be coupled with a calculator and a memory. The command center then makes it possible to deliver instructions to a command member, to calculate state variable values to generate different alerts and to store configuration information.

The body 2 of the catheter 1 preferentially has a rod shape.

The body 2 extends longitudinally up to its distal end 14. The body 2 is flexible or elastically deformable. A flexible body advantageously makes it possible not to damage the tissues of the premature baby, notably the tissues of the respiratory tracts and the tissues of the pharynx 202. According to an example, the body 2 of the catheter 1 comprises a sufficient stiffness to enable a guiding of the latter from an actuator and a sufficient flexibility making it possible to curve said body in order that it can move about in the throat, larynx, pharynx then if necessary beyond the vocal chords to and through the trachea. It is considered that the body 2 of the catheter 1 is deformable from the stage where its curvature is modifiable for example elastically.

The outer diameter of the body 2 must be sufficiently small for use in premature babies. The diameter of the body 2 must be sufficiently small for its insertion into the trachea through the vocal chords of a subject, notably a premature subject. According to one embodiment, the outer diameter of the body 2 is suited to be inserted into a conventional intubation probe. Advantageously, the outer diameter of the body 2 is comprised between 5 mm and 1 mm, preferentially between around 3 mm and 2 mm. A diameter less than 3 mm or less than 2.5 mm makes it possible to reserve the possibility of also using the catheter on a premature newborn baby already intubated with an endotracheal probe without accessory channel. Indeed, the diameter of the trachea of a premature newborn baby may go down to 2 or 3 mm. The premature newborn baby can thus benefit from the spray of medicine.

The length of the body 2 is preferentially less than 50 cm or comprised between 10 cm and 30 cm.

The body 2 extends from a proximal end (not represented) up to a distal end 14.

The body 2 of catheter 1 is preferentially designed to be single use. Preferentially, the catheter body is designed to receive an optic fiber and to be able to remove this optic fiber before throwing away the body 2 of catheter 1.

The invention also relates to a medical system 300. The medical system comprises a command unit 100. The command unit 100 is designed to be connected to the catheter 1 according to the invention. The command unit advantageously comprises a user interface enabling the control of the catheter 1. Once used, the catheter 1 or the catheter body may be disconnected from the command unit 100 and thrown away. In this case, a new catheter 1 is next connected to the command unit during a new use.

The body 2 comprises several conduits or lumens in its volume. The conduits or lumens extend into the longitudinal direction of the body.

The conduits or lumens extend preferentially up to the distal end 14 of the body 2 and/or preferentially from the proximal end of the body 2.

Preferentially, the distal end 14 of the catheter 1 comprises a substantially rounded shape. The substantially rounded shape advantageously enables the catheter to reduce the risk of trauma of the subject during the insertion of the catheter.

### Passage Channel

The body 2 comprises a first passage lumen 31. The catheter 1 comprises a channel for passage 3 of a liquid, arranged at least in part in the first lumen 31.

The passage channel 3 is designed to transport a medicinal liquid from a reservoir. The proximal end of the passage channel 3 is, in this case, preferentially connected to a reservoir of medicinal liquid. The medicinal liquid may be replaced by a medicinal powder, a gel or a paste.

According to an example, the passage channel 3 comprises a tubing arranged inside the passage lumen 31. In an alternative embodiment, the passage channel 3 is a conduit formed by the passage lumen 31. The surface of the passage lumen comprises, for example, an inner coating to facilitate the transport of the medicinal liquid. The inner coating may also make it possible to chemically protect the medicinal liquid from the walls of the lumen 31.

In a first embodiment, the passage channel 3 comprises a moveable element 5. The moveable element 5 is capable of transporting the medicinal liquid in its inner volume. The moveable element 5 translationally moves with respect to the body 2 of the catheter 1. The translation can preferentially be commanded from the proximal end by means of a drive member.

The moveable element 5 is designed in such a way as to discharge the medicinal liquid through a distal orifice 501 in the form of microdroplets. The distal orifice 501 of the moveable element 5 is fluidically connected to the passage channel 3.

The moveable element 5 preferentially comprises a distal end designed to reduce the trauma of the subject in the event of contact between the respiratory tracts and the distal end of the moveable element 5. Preferentially, the distal end of the moveable element comprises a rounded or curved portion.

### Translation of the Distal Part

The moveable element 5 of the passage channel 3 is translationally free with respect to the end distal 14 to the body 2 or with respect to an optic 6 of the body 2.

Preferentially, the moveable element 5 translationally moves beyond the body 2 up to a translated position. The moveable element moves beyond the body 2 of catheter 1 over at least 5 cm, very preferentially over a distance greater than 0.5 cm or 1 cm. In one embodiment, the moveable element 5 can be driven over a range of positions beyond the body 2 comprised between 0.5 cm and 5 cm. In one embodiment, in its most translated position, the distal end of the moveable element 5 is at a distance, counted from the distal end 14 of the body, comprised between 0.5 cm and 2 cm or between 0.5 and 4.5 cm.

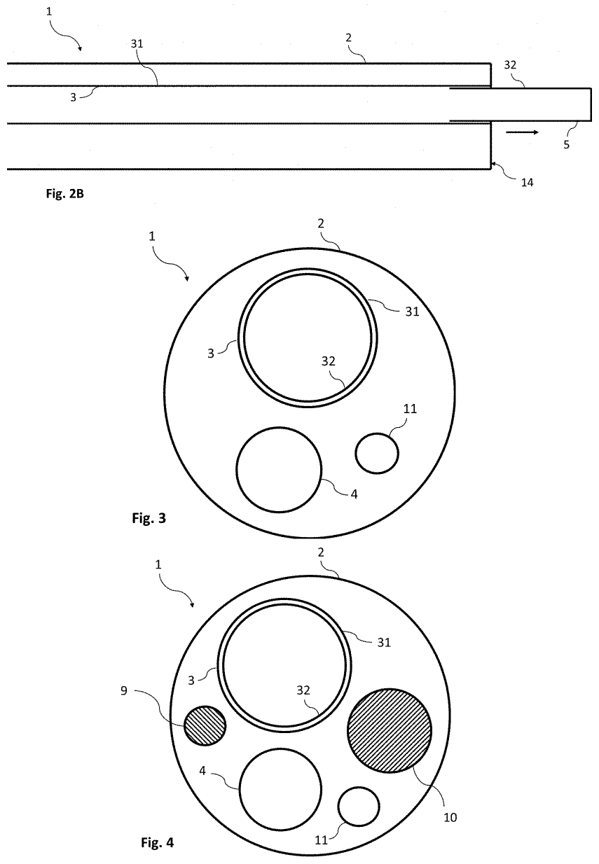

As illustrated in FIGS. 2A and 2B, the moveable element 5 of the passage channel 3 is displaced beyond the end 14 of the body 2 of the catheter 1.

According to one embodiment, the diameter of the moveable element 5 of the passage channel 3 is smaller than the diameter of the body 2 of catheter 1. This solution makes it possible to integrate the moveable element 5 within the passage channel 3. One advantage of the free translation of the moveable element 5 is to be able to penetrate into the trachea 203 through the vocal chords 204 of the premature baby 200. Free translation is understood as free mechanical translation, but being able to be activated and commanded by a command of an operator. Advantageously, a predefined distance may be preconfigured. According to another example, the operator deploys the moveable part while following step by step the progression, notably thanks to the optic arranged on the catheter. Another advantage is to be able to make the moveable element 5 advance from the passage channel 3 into an image capture zone of the optic 6. The driving of the translation is thus facilitated and the control in real time of the delivery of the medicinal solution is ensured.

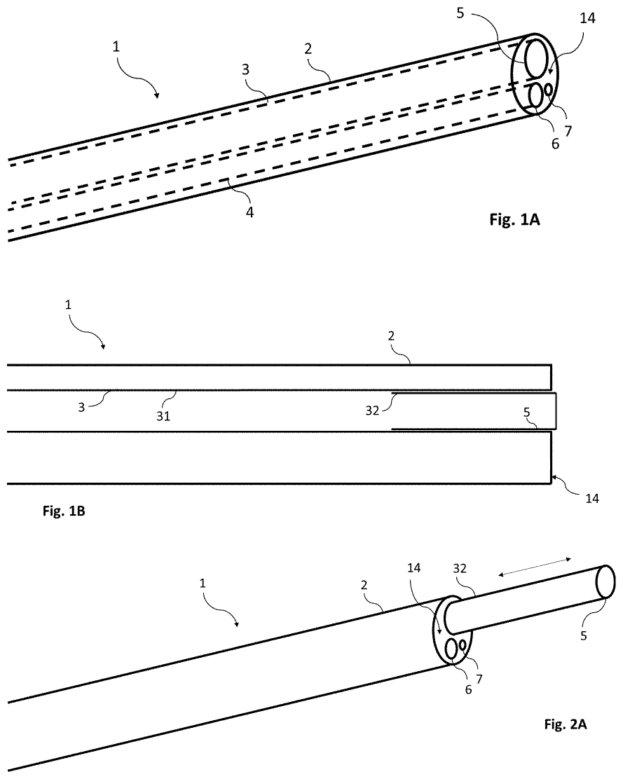

The moveable element 5 is moveable at least between a retracted position inside the body 2 of the catheter 1 (see FIGS. 1A and 1B) and a translated position where at least one portion of the moveable element 5 extends beyond the passage lumen 31 and the catheter body 2 (see FIGS. 2A and 2B).

Another advantage is to be able to conserve the moveable element 5 of the passage channel 3 in retracted position to reduce the risks of breakages or deformations of the moveable element 5 during the insertion of the body 2 of catheter 1 into the respiratory tracts of the subject 200.

In one embodiment illustrated in FIGS. 1B and 2B, the passage channel 3 comprises at least one portion concentric 32 with the passage lumen 31. The concentric portion 32 comprises the moveable element 5.

The concentric portion 32 is translationally moveable inside the passage lumen 31.

In one embodiment, not represented, the passage channel 3 comprises at least two telescopic concentric portions 32.

The telescopic concentric portions are translationally moveable in the passage lumen and with respect to one another. The concentric portions advantageously enable the increase of the protrusion length of the moveable element and the stiffness of the part of the passage channel outside the body 2 of the catheter 1.

### System for Displacing the Passage Channel

The catheter 1 may comprise a first member for driving the translation of the moveable element 5. The first drive member may comprise a control rod. Preferentially, a control rod (not represented) is connected to the concentric portion 32. The control rod may extend the proximal end of the body 2 of the catheter 1. The control rod allows the practitioner to displace the concentric portion 32 by actuating said control rod.

The first drive member makes it possible to command the movement of the moveable element 5. The first drive member makes it possible to move the moveable element and/or the concentric portion 32 beyond the catheter body of the means for activating the displacement of the passage channel 3 relatively to the body 2 of the catheter 1 in the distal direction.

For example, if the passage channel 3 comprises several telescopic concentric portions 32, the first drive member makes it possible to translationally move the telescopic portions with respect to one another in order that the moveable element projects from the body 2 of catheter 1.

The command unit 100 preferentially comprises a first command member. The command member is connected to the first drive member. The command member makes it possible to control the first drive member and the displacement of the distal part 5 of the passage channel 3. To this end, indicators of the position or deployment of the catheter may be displayed. According to an exemplary embodiment wherein the distal part of the catheter comprises a force feedback sensor, the command member can automatically stop the translational deployment of the moveable element when it encounters an obstacle such as the wall of the throat or the trachea, a vocal chord or any other portion of organ. The command unit 100 may in this case emit a sound or an alert informing the operator.

In an alternative embodiment, the spray device 50 is fixed with respect to the distal end 14 of the catheter body. The spray device 50 may be arranged at the distal end of the passage channel 3 in such a way as to spray in the extension of the distal end of the catheter body. This layout also makes it possible to supply the spray device through the lumen of the passage channel. In this embodiment, the catheter body 2 is designed to be able to be inserted into the trachea through the vocal chords of a subject, preferentially of a premature baby subject.

### Spraying of Microdroplets

The moveable element 5 comprises a spray device 50.

In one embodiment, the spray device 50 is designed to spray a powder, notably a medicinal powder.

In a second preferential embodiment, the spray device 50 is designed to expel a medicinal liquid in the form of microdroplets.

In a first alternative, not represented, the microdroplets of medicinal liquid are obtained by nebulization. The body 2 of catheter 1 then comprises a channel for the transport of a flow of pressurized gas. The catheter is then designed so that the pressurized gas encounters the medicinal liquid close to the distal orifice of the passage channel 3. The pressurized gas makes it possible to nebulize or to atomize the medicinal liquid into microdroplets.

The medicinal liquid is then sprayed into the trachea in the form of nebulized microdrops. In this first alternative, the body 2 of the catheter 1 comprises a lumen making it possible to convey the gas.

In a second alternative represented in FIG. 6, the microdrops of medicinal liquid are administered in the form of an aerosol. An aerosol is produced when a pressurized liquid passes through a conduit of which the outlet comprises a particular shape favorable to the creation of microdrops.

In a particular example illustrated in FIG. 6, the spray device 50 of the passage channel 3 comprises aerosolization means 502, 503, 504, 506, 501 for expelling the medicinal liquid in the form of an aerosol.

The aerosolization means comprise an insert 502 generally elongated and arranged inside the passage channel 3.

The insert 502 comprises an outer surface. Said outer surface comprises at least one furrow 503 winding around the outer surface of the insert 502. The at least one furrow 503 is suited for the passage of the medicinal liquid. The furrow 503 is preferentially helicoidal. The at least one furrow 503 extends from the proximal end up to the distal end of the insert 502.

The summits of the outer surface of the insert, between two successive passages of the at least one furrow 503 are in contact with the inner surface 508 of the passage channel 3 or form a leak tightness for forcing the medicinal liquid to follow the at least one furrow 503. The medicinal liquid thus passes in the furrows 503 between the outer surface of the insert 502 and the inner surface 508 of the passage channel 3.

The aerosolization means preferentially comprise in the passage channel 3 a receiving chamber 505. The receiving chamber is arranged at the outlet of a furrow 503 of the insert 502 and the end. Preferentially, the receiving chamber is arranged between the insert and the distal end of the spray device 50.

The spray device 50 comprises a distal orifice 501, preferentially at the distal end of the spray device 50.

The aerosolization means also comprise an outlet channel 509. The outlet channel 509 extends into the extension of the receiving chamber. The outlet channel 509 is suited for the passage of the medicinal liquid. The outlet channel 509 enables the pressurization of the medicinal liquid, notably by its profile. In one embodiment, the profile of the channel comprises at least one first portion, connected to the receiving chamber, the section of which decreases in the distal direction. The profile of the outlet channel 509 comprises a second portion, connected to the distal orifice 501, the section of which is substantially constant. In one embodiment, the first portion is adjacent to the second portion.

In one embodiment, the outlet channel 509 is formed by a profiled body 504. The profiled body 504 is arranged inside the passage channel 3 between the distal end 501 of the passage channel 3 and the insert.

The diameter of the distal orifice 501 is preferentially comprised between 20 μm and 100 μm, very preferentially between 40 μm and 80 μm. In one embodiment, the diameter of the distal orifice 501 is comprised between 20 μm and 250 μm, notably when the volume of microdroplets to produce is greater.

When the medicinal liquid in the passage channel 3 is pressurized by a pump, the liquid is forced to follow the helicoidal furrow 503 between the outer surface of the insert 502 and the inner surface 508 of the passage channel 3. The furrow 503 plays the role of swirl generator. At the outlet of the helicoidal furrow 503, the medicinal liquid penetrates into the receiving chamber 505 and the direction of flow is substantially a circular path following the inner circumference of the passage channel 3.

At the distal end of the chamber 505, the rotating liquid encounters the outlet channel 509 in which is created an interface between the swirling liquid in the receiving chamber 505 and the ambient atmosphere of the orifice 501. The generation of the aerosol then takes place at the distal end of the orifice 501 characterized by the ejection of microdroplets.

The shape of the aerosol may be modified as a function of the pression exerted on the medicinal liquid, the angle of rotation of the liquid in the chamber, the geometry of the chamber and the geometry of the body and of the distal orifice.

The spray device 50 may further comprise a stop 506 limiting the translational movements of the insert 502.

The spray device 50 may further comprise a stop 507 limiting the translational movements of the profiled body 504.

The stops 506, 507 advantageously make it possible to maintain in position in the passage channel 3, the insert 502 and the body 504 despite the pressure of the medicinal liquid in the distal direction of the passage channel 3.

In one embodiment, the moveable element comprises several spray devices in parallel. The increase in the number of spray devices 50 makes it possible to increase the flow rate of medicinal liquid expelled by microdroplets. The catheter may also comprise several moveable elements 5 in parallel each comprising a spray device 50.

### Administration of the Surfactant

The medical system 300 comprises a reservoir of medicinal liquid intended to be administered to the subject. The reservoir is fluidically connected to the passage channel 3. The medicinal liquid preferentially comprises a surfactant. The surfactant may preferentially comprise a composition comprising a diluted pork lung extract. Alternatively, an artificial surfactant may be used. Generally speaking, any surfactant enabling the efficient treatment of respiratory distress syndrome may be used.

Preferentially, the reservoir of medicinal liquid comprises means for heating the medicinal liquid. One advantage is to reduce the viscosity of the medicinal liquid, facilitating its passage through a passage channel 3 of low diameter, thus improving miniaturization of the catheter. Another advantage is to deliver into the respiratory tracts of the subject a medicinal liquid closer to the body temperature of the subject. The medical system 300 may comprise for this purpose temperature regulating means to reach a target temperature value of the medicinal liquid in the reservoir.

In one embodiment, not represented, the reservoir comprises a syringe. The heating means may then comprise a heating band, arranged around the reservoir of the syringe.

The reservoir is fluidically connected to a pump. The pump is preferentially a high pressure pump. The pump advantageously makes it possible to pressurize the medicinal liquid inside the passage channel 3. The high pressurization of the medicinal liquid inside the passage channel makes it possible to generate an administration in the form of an aerosol. High pressure is taken to mean a pressure comprised between 80 bars and 200 bars, preferentially between 100 bars and 180 bars, very preferentially between 120 bars and 160 bars. Alternatively, the pressure may be comprised between 80 bars and 350 bars, particularly for the most important diameters of distal orifice.

The pump is connected to a device for commanding the pump. The device for commanding the pump makes it possible to command the activation of the pump and its intensity.

In one embodiment, the command device of the pump may be commanded by the practitioner. Preferentially, the command device of the pump comprises a “pistol” type trigger or another type of command.

In an alternative embodiment, the reservoir comprises a medicinal powder. The medicinal powder preferentially comprises a surfactant. In particular, the surfactant may comprise a composition comprising a diluted pork lung extract. Alternatively, an artificial surfactant may be used. Generally speaking, any surfactant enabling the efficient treatment of respiratory distress syndrome may be used.

The invention also relates to a medical system and a catheter of which the channel for passage 3 of the medicinal liquid is fixed with respect to the catheter body. In this embodiment, the moveable element is not translationally free or does not exist. The spray device 50 for expelling the medicinal liquid in the form of microdroplets is the same as that described previously. In this embodiment, the catheter body comprises a sufficiently small diameter to enable its passage through the vocal chords. Preferentially, the diameter of the catheter body is less than 2.5 mm for an application on the newborn or less than 5 mm for an application on an adult subject.

### Respiratory Cycle Measurement

In one embodiment, the medical system 300 comprises a device for measuring the respiratory cycle of the subject 200. The device for measuring the respiratory cycle of the subject 200 comprises, for example, sensors of the respiration of the subject and/or a sensor of the enlargement of the thoracic cage.

The device for measuring the respiratory cycle of the subject 200 may comprise an air sensor or a change of air flow rate sensor. The sensor may be arranged at the level of the airways of the subject, for example at the level of the nasal or mouth passages of the subject. Alternatively, the sensor may be arranged on a channel connected to the airways of the subject.

In another embodiment, the device for measuring the respiratory cycle of the subject 200 may comprise at least one electrical impulse sensor. The electrical impulse sensor is arranged in such a way as to capture the electrical impulses of the respiratory muscles, in particular the diaphragm. Such a sensor advantageously makes it possible to detect the electrical signals causing an activity of the muscle, and thus a phase of inspiration or expiration in advance.

The device for measuring the respiratory cycle of the subject 200 may also comprise an optic (not represented) enabling the detection of phases of inspiration and expiration of the subject.

Preferentially, the command device is configured to administer the medicinal liquid in synchronization with the respiratory cycle of the subject. The command device is then connected to the device for measuring the respiratory cycle of the subject 200. For example, the command device may be configured to command the administration of the medicinal liquid uniquely during phases of inspiration of the respiratory cycle of the subject.

This synchronization advantageously makes it possible to favor the transport of the medicinal liquid in the form of microdroplets to the lungs thanks to the respiration of the subject and to decrease its expulsion by the respiratory tracts out the trachea.

In an alternative embodiment, the spray device may be replaced by a collection device, notably by suction or by a device for delivering a liquid or a paste or a gel.

### Camera

The body 2 of the catheter 1 comprises an optic 6. The optic is preferentially configured or arranged to generate an image of the distal end of the moveable element 5 in translated position.

The optic may comprise a camera or a micro-camera.

The optic preferentially comprises an optic fiber. The optic fiber has a distal end arranged on or close to the distal end 14 of the body 2.

Preferentially, the optic is configured or arranged to generate an image of the distal end of the moveable element 5 in translated position or at the level of the distal end of the moveable element 5 in translated position.

In one embodiment, the optic 6 is arranged close to or at the level of the distal end 14 of the body 2. The body 2 then comprises a second lumen 4 for the passage of a means for connecting the optic 6, such as an electric cable or an optic fiber.

Preferentially, the optic 6 is arranged to capture images of a zone facing the distal end 14 of the body 2. The optic 6 is arranged to capture images of a zone in the longitudinal direction of the body 2 of the catheter 1. In this way, the practitioner can advantageously visualize the glottis of the subject during the progression of the catheter in the passages of the subject. This visualization advantageously makes it possible to gain precious intervention time, notably during a procedure for treating the subject as a matter of urgency. Once the catheter body has passed through the vocal chords, the optic also makes it possible to visualize the bifurcation between the two bronchi sources (also called carina) in order to place the distal end of the catheter at an appropriate distance from this bifurcation.

The lumen 4 for the passage of an optic fiber, designated optic fiber lumen, extends longitudinally inside the body 2 of the catheter 1. The optic fiber lumen 4 may extend from the proximal end of the body 2, up to the distal end 14 of the body 2 or up to the optic 6.

The proximal end of the optic fiber is coupled to a signal processing device for the generation of an image from the signals of the optic fiber.

The optic fiber has the advantage of being less voluminous than a camera, the device for processing the luminous signal being arranged outside of or at the proximal end of the body 2.

In an alternative embodiment, not represented, the optic fiber extends against the outer wall of the body 2 of the catheter. The optic fiber may then be maintained against the wall by different fastening means, such as a rail, a groove or a rib realized on the surface of the catheter body 2.

In another alternative embodiment, not represented, the body 2 comprises wireless transmission means for the transmission of the images taken by the optic 6.

The catheter 1 may comprise means for orienting the shot zone of the optic 6. These orientation means can preferentially be commanded remotely. The orientation means make it possible to modify the orientation of the optic 6 while the catheter 1 is in use. To this end, the optic 6 may be mounted on a pivot or swivel link.

The catheter 1 may also comprise means for displacing the optic 6 with respect to the body 2. The optic may be capable of translationally moving with respect to the body 2 of the catheter 1. To this end, it may be integral with a rod moving (or capable of moving) in translation.

The optic is preferentially configured to acquire images in the visible range. In alternative embodiments, the optic may be monochromatic. It may be configured to acquire images in black and white or for example in a range of infrared frequencies.

In a preferential embodiment, the optic fiber is translationally moveable in the lumen 4 for the passage of the optic fiber. This mobility advantageously makes it possible to use the catheter for a single use while conserving the optic fiber for a later use in a second catheter according to the invention.

In this embodiment, the lumen 4 for the passage of the optic fiber comprises, at its distal end, a leak tight wall. The leak tight wall makes it possible to isolate the optic fiber and the lumen 4 for the passage of the optic fiber from the external environment of the catheter. The leak tight wall makes it possible to protect the optic fiber from contamination and advantageously allows re-use of the optic fiber without having to decontaminate it between two uses. The leak tight wall also guarantees the sterility of the optic fiber. The leak tight wall is preferentially transparent to enable image capture by the distal end of the optic fiber. “Transparent” is here taken to mean transparent to the light rays captured by the optic. These rays may thus comprise rays in the visible domain, infrared rays or other.

### Lighting

Preferentially, the catheter 1 comprises a light source 7. The light source 7 is arranged in such a way as to light up at least one part of the zone captured by the optic 6. The light source 7 may comprise one or more light emitting diodes. The light source 7 is connected to a supply cable. The body 2 of catheter 1 may comprise a third lumen 11 made inside the body 2. The supply cable of the light source 7 is then arranged at least in part inside the third lumen 11. According to one embodiment, the lumen making it possible to convey an electric cable or an optic fiber may be shared to convey the electrical supply of the light source.

The light source 7 advantageously makes it possible to emit light to light up the zone captured by the optic 6 and to allow the practitioner to visualize the zone in front of the distal end 14 of the body 2 of the catheter. The guiding of the catheter body inside the larynx 206 is thus facilitated.

The light source 7 is designed to emit light detectable by the optic 6. For example, the light source 7 may comprise an infrared light source if the optic 6 is an infrared camera.

In a preferential embodiment, the light source is the optic fiber for the image capture. The optic fiber then plays the two roles of optic for image capture and of light source. One advantage is to avoid the creation of an additional lumen in the catheter body 2 for the passage of a light source, thus increasing the miniaturization of the catheter. Another advantage is to reduce the number of manipulations before throwing away the single use catheter.

The optic fiber may be used in such a way that a first radial portion of its section is configured to emit light and a second radial portion to capture images. For example, the outer ring of the section of the optic fiber is used as light source. For this purpose, suitable means are provided at the level of the proximal end of the optic fiber. In this example, the portion of section inside the outer ring is used as image capture.

### System for Controlling the Optic Means

According to one embodiment, the medical system 300 comprises, further, a device for controlling the optic. The device for controlling the optic makes it possible to control the optic 6 and/or the activation of the light source 7.

The medical system may comprise an information transmission means, notably a display screen 101. The display screen 101 makes it possible to display in real time the images captured by the optic 6 of the catheter 1.

According to one embodiment, the user interface comprises an interface for commanding optic means. The command interface comprises a command for the activation and/or the adjustment of the intensity of the light source 7 and/or of the optic 6. The command interface of the optic means is configured to place at the disposal of an operator a control of the orientation and/or of the displacement of the optic 6. This control is ensured, for example, by a numerical command. Alternatively, the control is ensured by a mechanical command, for example, by means of a handle.

According to one exemplary embodiment, the display device generates numerical indications superimposed on the acquired image. These indications are for example organ contour lines, such as the vocal chords. These contour lines may be generated from a shape recognition algorithm. One interest is to improve the reading of information which has to be done in a restricted time interval for the operator. According to another example, indicators of distance, orientation are displayed in order to improve the legibility of the situation underway. It may be, for example, the translational distance of the moveable element or the orientation of the distal part of the catheter, or even the deflectable portion. According to another example, indicators are displayed superimposed on the acquired image indicating intervention times, indications of volumes inspired/expired by the subject, etc.

### Deflectable Portion

The body 2 of the catheter comprises a second drive member. The second drive member makes it possible to drive an orientation of a distal part 81 of the body 2 of catheter 1 with a proximal part 82 of said body 2 so as to control said orientation of the distal part 81 in a predetermined plane and/or in a predetermined direction.

In one embodiment, the catheter body 2 comprises at least one orientable portion 8. This orientable portion 8 is designed to be curved in a predetermined plane and/or in a predetermined direction. Preferentially, the orientable portion is designed to be curved in a controlled manner when this orientable portion is activated. The catheter 1 comprises activation means 9 to trigger and to control the folding or the curvature of the orientable portion 8.

“Controlled manner” is taken to mean that the practitioner is capable of adjusting the angle of curvature or the angle between the distal part 81 and the proximal part 82 of the catheter body 2 over a predetermined range. The practitioner can thus progressively increase or decrease the angle of orientation of the distal part 81 between two extremums in the predetermined plane.

Advantageously, the control of the angle of orientation of the distal part 81 makes it possible to insert the body 2 of the catheter 1 into the larynx 206 of the subject without use of a laryngoscope.

The maximum angle folding of the body in the predetermined plane is at least 45°, preferentially at least 70°.

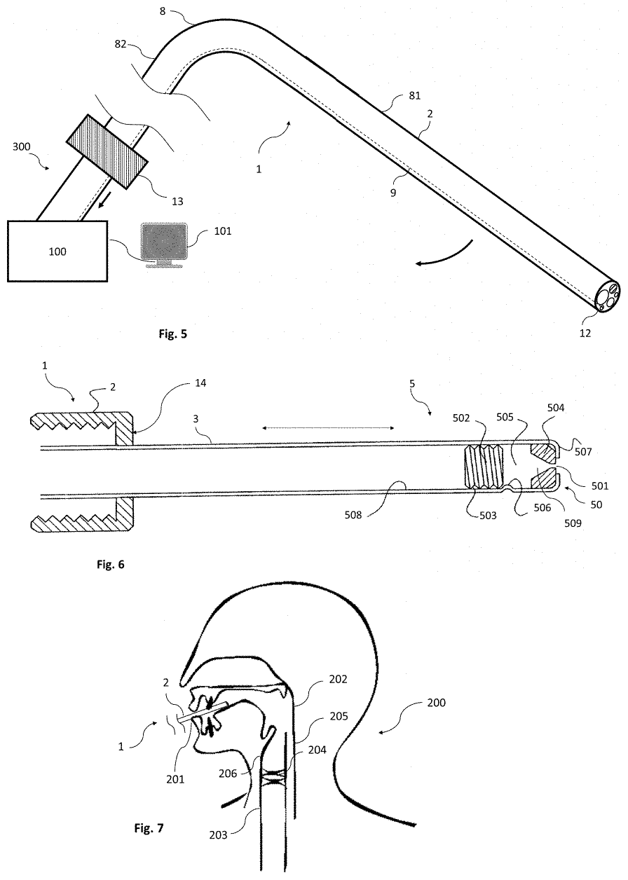

In one embodiment represented in FIGS. 4 and 5, the second drive member comprises an activating wire 9 arranged along the catheter, for example, inside the body 2 of the catheter 1.

The activating wire 9 may be arranged inside a longitudinal lumen of the body 2 of the catheter 1. Said longitudinal lumen and the activating wire 9 are preferentially radially off-centered with respect to the longitudinal axis of the body 2 of the catheter 1. This radial offset advantageously makes it possible to favor the change of orientation of the distal part 81 of the catheter body 1 in a predetermined plane. The predetermined plane then comprises the longitudinal axis of the body 2 of the catheter 1 and the longitudinal axis of the lumen comprising the activating wire 9.

According to one embodiment, the catheter 1 comprises an anchoring 12. The anchoring 12 is a zone at the level of which the activating wire 9 is translationally blocked with respect to the body 2 of catheter 1. At the least, the anchoring 12 translationally blocks the movement of the wire in the proximal direction with respect to the body 2 of the catheter 1.

According to an example, the anchoring 12 is arranged at the level of the distal end 14 of the body 2 of the catheter 1. According to an alternative, the anchoring 12 is done inside the body 2, between the distal end of the orientable portion 8 and the distal end 14 of the body 2.

The anchoring 12 is done, for example, by bonding or by crimping. The anchoring 12 may also comprise an obstacle, an obstruction or a node of the wire between the distal end of the body 2 and a retention means.

According to another embodiment, the activatable wire 9 is arranged along the outer surface of the body 2 of catheter 1. The outer surface then comprises passage means for the activatable wire 9. The passage means are preferentially fastened to the outer surface of the body 2 to avoid arc effects of the activatable wire during folding.

When the activatable wire 9 is drawn, a tractive force at the level of the anchoring 12 along the longitudinal direction of the body 2 is exerted in the proximal direction. This force advantageously makes it possible to fold the orientable portion and to create an angle of orientation between the distal part 81 and the proximal part 82 of the body 2 of catheter 1 in the predetermined plane.

In an alternative embodiment illustrated in FIG. 13, the second drive member comprises a preformed tube 9 in a lumen 20 of the catheter body 2. The preformed tube 9 extends into an orientation lumen 20 of the catheter body 2. The tube is preformed in such a way that it comprises a preformed portion which, when the tube is at rest, takes a curved shape, inducing an angle between the parts of the tube on either side of this portion in a predetermined plane. The second drive member also comprises a rigid rod 22 extending into the cavity of the preformed tube 9. The rigid rod 22 has a substantially rectilinear shape at rest. The stiffness of the rigid rod 22 is greater than that of the preformed tube 9. Preferentially, the rigid rod 22 extends from the proximal end of the preformed tube 9, at least up to the preformed portion or up to a point situated between the preformed portion and the distal end of the preformed tube 9. The rigid rod may be removable with respect to the preformed tube.

In this way, when the rod is inserted into the cavity of the preformed tube 9, the preformed tube 9 deforms to take the shape of the rigid rod 22. When the rigid rod 22 is removed, the preformed tube 9 then tends to recover its shape at rest and applies a force on the catheter body. This force advantageously makes it possible to fold the orientable portion of the catheter body and to create an angle of orientation between the distal part 81 and the proximal part 82 of the body 2 of catheter 1 in the predetermined plane as illustrated in FIG. 5. Preferentially, the preformed tube enables a deformation of the distal part of the catheter between 20 and 40°.

This embodiment advantageously makes it possible, by removing little by little the rigid rod from the preformed tube, to control the angle of folding of the distal end of the catheter body in the predetermined plane.

The preformed tube preferentially comprises a plastic or metal material. The rigid rod is preferentially a steel type metal.

The orientable portion 8 may comprise at least one zone of which the stiffness is less than the stiffness of the distal 81 or proximal 82 part of the body 2. This stiffness makes it possible to favor the appearance of a fragility zone and to favor the curvature on the orientable portion 8. The orientable portion 8 may comprise a stiffness less than the stiffness of the remainder of the body 2 uniquely on a lateral zone of the orientable portion 8. These lateral zones make it possible to favor advantageously the curvature of the body 2 in a predetermined plane comprising said lateral zone.

In one embodiment, the orientable portion 8 of the catheter body extends over a length comprised between 20 mm and 30 mm. The tip deflection thus advantageously makes it possible to facilitate the passage of the catheter 1 between the nasopharynx and the oropharynx.

The zones of lower stiffness may comprise a less rigid material than the material of the remainder of the body 2 or by cavities.

Preferentially, said lateral zone is comprised in the plane comprising the longitudinal axes of the body 2 and the lumen comprising the activatable wire 9. This configuration advantageously favors the folding of the orientable portion 8 in a predetermined plane during the activation.

In a first example, the activation is generated by the traction of the activatable wire 9. By a traction that is more or less strong, it is possible to control the orientation of the distal part 81 in the predetermined plane.

In a second example, the activatable wire 9 is a wire made of shape memory alloy. The catheter 1 then also comprises means for passing a current through the shape memory alloy wire. The activation of the shape memory alloy wire is carried out by applying a voltage to the wire. The application of a voltage makes it possible to increase the temperature of the wire by Joule effect until reaching a temperature of change of phase of the shape memory alloy wire.

The activation of the shape memory alloy wire triggers a predetermined curvature of the wire. The angle of orientation of the distal part 81 may then be controlled by the temperature or by the electric voltage applied to the shape memory alloy wire.

These examples do not limit the invention which may comprise any other means for triggering, by activation, a curvature controlled in a predetermined plane of the distal part 81 of the body 2 with respect to the proximal part 82. In one embodiment, the orientable portion 8 extends between 2 cm and 5 cm.

In one embodiment, the distal end of the orientable portion is arranged between 15 mm and 100 mm from the distal end of the body 2 of the catheter 1.

In one embodiment, the body 2 of the catheter comprises at least two second driving members making it possible to drive the orientation of the distal part 81 in at least two different predetermined planes/or in at least two different predetermined directions. This solution may be realized for example from two wires combined and each being activable in a given plane. According to another exemplary embodiment, the two associated wires may be with shape memory, said shape memory wires having different alloys and particular preforms.

Preferentially, the catheter comprises a mark to indicate to a user the direction of the predetermined plane in which the body 2 is going to deform. The mark may be made on a proximal part and/or on the outer surface of the catheter

### System for Activating the Orientable Portion

In one embodiment, the medical system 300 also comprises a means for actuating the drive member. The actuating means may be designed to exert a tractive force on the activatable wire 9. In the case where the activatable wire 9 is a wire made of shape memory alloy, the actuating means are designed to generate a tension over the length of the shape memory alloy wire.

The actuating means also make it possible to control the angle of curvature of the orientable portion 8 by adjusting the tractive force or the intensity of the current imposed on the activatable wire 9.

The user interface may comprise an interface for commanding the orientation of the distal part 81 of the body 2 of the catheter 1. This interface is connected to the actuating means and commands said actuating means and makes it possible to command the triggering of the curvature of the orientable portion and to control this curvature.

When the drive member comprises a preformed tube and a rigid rod as described above, the actuation of the drive member may be carried out by the removal of the rigid rod. The removal is preferentially carried out by the proximal end of the preformed tube or the catheter body.

The drive member and the optic advantageously allow a rapid insertion of the distal end 14 into the larynx of the subject through the epiglottis. Indeed, the practitioner can easily locate the junction between the larynx and the esophagus and activate the curvature of the body up to said junction to make the body 2 penetrate into the larynx.

### Ventilation

During the administration of the medicinal liquid to a premature baby, it is very often necessary to assist the respiration of the premature baby.

The body 2 of the catheter 1 may be designed to be inserted at least in part into an intubation probe. Respiration assistance may also be done by a mask or a probe inserted through the nose independently of the catheter 1.

In this embodiment, the catheter body 1 may be easily used on a patient already intubated by inserting the catheter 1 through the intubation probe. The invention thus relates to an intubation probe suited for receiving said catheter 1. The adaptation may be achieved by an element inside the intubation probe having guiding ergonomics or instead a physical means intended to guide the movements of the catheter 1 within the probe. The intubation probe comprises at its distal end means for being connected to a ventilation device such as a respirator or any other respiratory aid device.

When a catheter is introduced into the larynx of a subject and used to deliver a spray, another advantage of a probe being able to be coupled to a catheter of the invention is to be able to rapidly intubate the subject. Indeed, in this case, the intubation probe may be positioned upstream of the catheter and free to slide along the body 2 of the catheter 1. This option is particularly interesting in the event of cardiac and/or respiratory anomalies of the subject. The use of a laryngoscope is then unnecessary since the catheter 1 is already in the larynx of the subject.

In an alternative, the body 2 of the catheter 1 may comprise a ventilation lumen 10 in its inner volume. The ventilation lumen 10 is connected to a ventilator, an air pump or to a respiratory support system, preferentially by its proximal part. The ventilation lumen 10 preferentially extends up to the distal end 14 of the catheter body 2.

The ventilation lumen 10 advantageously makes it possible to integrate a system for assisting ventilation with the catheter 1. The practitioner caring for the premature baby then has fewer operations to carry out before beginning the administration of the medicinal liquid. The ventilation lumen 10 advantageously makes it possible to maintain a respiratory support to the subject during the administration of the medicinal liquid. The ventilation lumen 10 also enables the introduction of a suction pipe through said lumen, for example to suck up secretions of the subject.

### Ventilation System

In the embodiment where the catheter 1 comprises a ventilation system, the device for measuring the respiratory cycle of the subject is connected to the ventilator. The ventilator is connected to an intubation probe or to the ventilation lumen 10. Preferentially, the air pump of the ventilation system is connected to the means for measuring the respiratory cycle. The ventilation system is configured to operate in synchronization with the respiratory cycle of the subject. This synchronization advantageously makes it possible to assist the respiration of the subject and to favor the delivery of microdrops into the lungs of the subject 200, more particularly into the alveoli of the lungs of the subject 200.

### Positioning Piece

In one embodiment, the body 2 of the catheter 1 comprises a positioning piece 13. The positioning piece 13 is designed to cooperate with the mouth 201 of the subject 200. The positioning piece may be designed to be in part or totally introduced into the mouth 201 of the subject 200.

The positioning piece 13 extends radially at least partially around the body 2 of the catheter 1.

The positioning piece 13 forms a buccal support. The positioning piece 13 is designed to limit the volume of air transiting outside the catheter 1 between the inside of the mouth 201 and the outside of the mouth 201.

The positioning piece 13 may be designed to be placed on the mouth 201 of the subject 200 or inside the mouth 201 of the subject 200.

Said positioning piece 13 makes it possible to ensure an at least partial leak tightness through the mouth of the subject. This leak tightness advantageously makes it possible to improve the efficiency of the respiratory support system during the administration of the medicinal liquid independently of whether the respiratory support system passes through the nose of the subject 200 or through the catheter 1.

In one embodiment, the positioning piece 13 comprises a guide to orientate the introduction of the catheter 1 into the respiratory tracts of the subject 200. The guide may comprise the inner walls of the positioning piece 13. The walls are intended to be in contact with the surface of the body 2 of catheter 1.

The positioning piece 13 acts as a pivotal point. This pivotal point advantageously makes it possible to facilitate the guiding of the catheter body by the practitioner.

The positioning piece 13 may be arranged to slide along the body 2 of the catheter 1. Once the positioning piece 13 cooperates with the mouth 201, the body 2 is translationally moveable with respect to said piece. The translational freedom advantageously makes it possible to improve the guiding of the body 2 during the insertion thanks to the pivotal point constituted by said positioning piece 13 in cooperation with the mouth 201.

Said positioning piece 13 is preferentially made of material making it possible to facilitate the leak tightness of said positioning piece 13. The positioning piece 13 may be made of plastic or elastic material compatible with use in contact with patients. In one embodiment, the positioning piece is made of expansible material. The shape of the positioning piece 13 may be designed in such a way as to make said piece 13 enter into the mouth 201 of the subject and to remain in the mouth 201 of the subject 200 during the intervention.

In one embodiment, the positioning piece 13 comprises a stop 131 on the proximal portion of the positioning piece. The stop 131 of the positioning piece 13 advantageously makes it possible to prevent the subject from swallowing said positioning piece. Another advantage of the stop 131 of the positioning piece 13 is to facilitate the translation of the body 2 with respect to the positioning piece 13.

In one embodiment, the positioning piece 13 is designed to allow a translation inside an intubation probe. Indeed, once the distal end 14 of the body 2 is in the larynx 206, it may be necessary to be able to introduce rapidly an intubation probe in the event of discomfort of the subject. In practice, the intubation probe is made to slide around the catheter. The positioning piece 13 thus allows such a sliding and is designed to enable the passage of air through the intubation probe.

In one embodiment, the positioning piece 13 is included in a mask intended to be mounted on the head of the subject in such a way as to isolate in a leak tight manner the mouth and the nose from the external environment. The mask may comprise an orifice for the passage of the catheter according to the invention in a leak tight manner.

### Command Center

The command center 100 preferentially comprises one or more of the following elements:
a reservoir for receiving a volume of a medicinal solution or a volume of medicinal powder;
a device for commanding the administration of the medicinal liquid driving a high pressure pump;
the ventilation device for assisting the respiration of the subject;
the device for measuring the respiratory cycle of the subject;
the actuating means for controlling the angle of curvature of the orientable portion 8;
the interface for commanding the orientation of the distal part 81;
the optic control device.

The command center 100 may comprise a user interface. According to different embodiments, the command center may be coupled with a calculator and a memory. The command center then makes it possible to deliver instructions to a command member, to calculate state variable values to generate different alerts and to store configuration information.

The command center 100 may comprise isolation means. The isolation means advantageously make it possible to avoid microbial contamination of the command center, notably between two interventions after having replaced the catheter.

The medical system 300 preferentially comprises a connection device enabling a reversible connection between the catheter 1 and the command center 100.

### Embodiments of the Invention

FIGS. 7 to 11 illustrate a method for administering a medicinal liquid by the use of a medical system 300 or a catheter 1 such as described above.

In a first step illustrated in FIG. 7, the distal part 82 of the body 2 of the catheter 1 is inserted into the mouth 201 of the subject 200. The insertion is performed by the practitioner.

The practitioner can then make the positioning piece 13 cooperate with the mouth 201 of the subject 200. The positioning piece is going to make it possible to improve the efficiency of the ventilation system. The positioning piece 13 is also going to make it possible to help the practitioner to guide the body 2 by creating a support or a pivotal point. The body 2 is pushed by the practitioner to progress through the pharynx 202.

In a second step, the practitioner, through the user interface, activates the member for driving the orientation of the distal part 81. The orientable portion 8 then begins to curve in the predetermined plane. The distal part 81 takes an orientation in a predetermined plane with respect to the proximal part 82.

As the body 2 folds, the practitioner can control the position of the distal end 14 with the screen 101. The practitioner can adjust the orientation of the distal part until he visualizes the entrance of the larynx 206 or the entrance of the esophagus 205 on the screen 101. Once the entrance of the larynx 206 visualized, the practitioner can introduce the distal end 14 of the body 2 into the larynx 206.

Once the distal end 14 of the body 2 introduced into the larynx 206, the practitioner can deactivate the second drive member. The curvature imposed on the orientable portion is relaxed. The body thus recovers its flexibility and can progress into the larynx 206 by a push on the body 2 of the catheter 1.

The means for driving the orientation of the distal part 81, the optic 6 and the display means advantageously make it possible to guide the distal end 14 of the body 2 of catheter 1 into the larynx 206 of the subject without using a laryngoscope and while minimizing the pain of the subject 200.

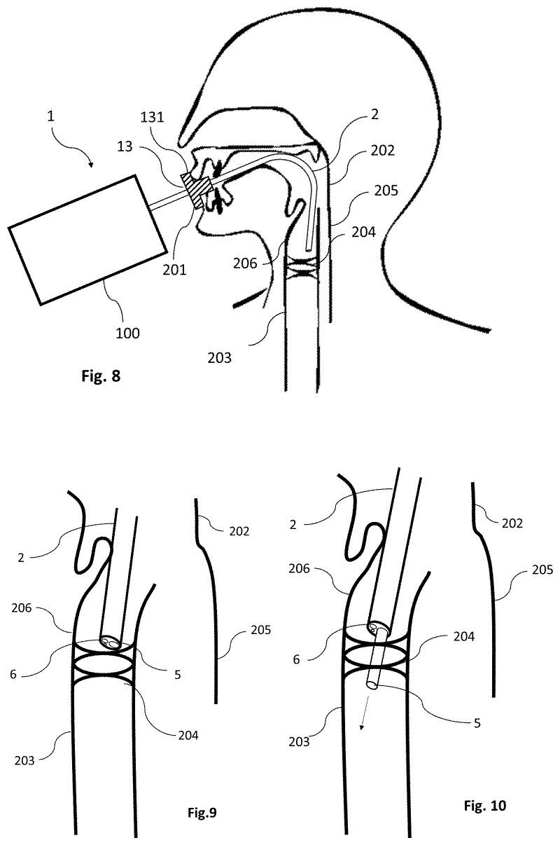

As illustrated in FIGS. 8 and 9, the body 2 of catheter 1 is advanced into the respiratory tracts until the distal end 14 of the body 2 of the catheter 1 arrives in front of the vocal chords 204 of the subject 200.

The practitioner can control the advancement of the catheter 1 up to the entrance of the vocal chords 204 through the screen 101 displaying the images taken by the optic 6.

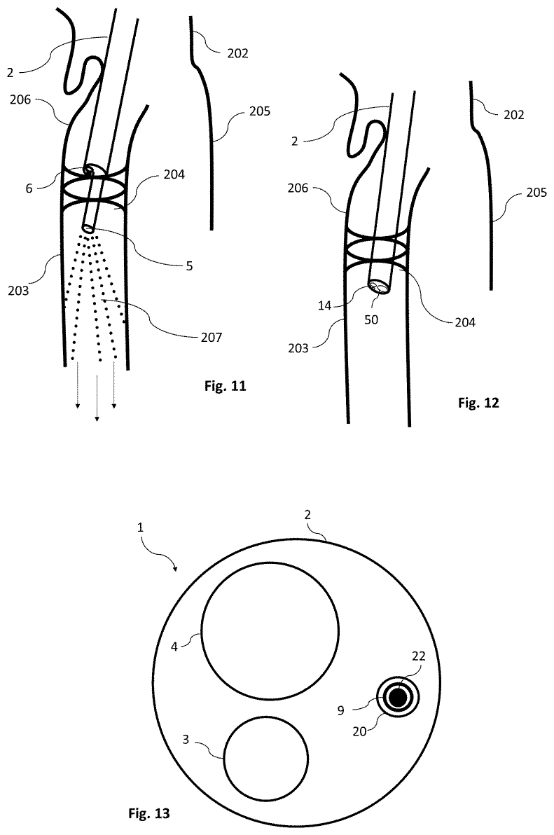

In a first embodiment illustrated in FIG. 10, once the distal end 14 of the body 2 of the catheter 1 is facing the vocal chords 204, the moveable element 5 of the passage channel 3 is translationally advanced beyond the body 2 of the catheter 1. The moveable element 5 is advanced until the distal end of the moveable element 5 passes through the vocal chords as illustrated in FIG. 12. Preferentially, the distal end of the moveable element 5 is advanced until in the trachea 203 of the subject 200. The optic 6 is arranged to capture images of a zone comprising the vocal chords 204 and the moveable element 5 of the passage channel 3. Using the screen 101, the practitioner can thus guide the moveable element between the two vocal chords.

As illustrated in FIG. 11, once the moveable element 5 is arranged in the trachea 203, the practitioner can administer the medicinal liquid in the form of microdroplets 207 into the trachea 203 of the subject through the distal orifice 501.

In a second alternative embodiment illustrated in FIG. 12, the distal end 14 of the catheter body 1 is advanced to make the catheter body pass through the vocal chords 204.

The optic 6 is arranged to capture images of a zone comprising the vocal chords 204. Using the screen 101, the practitioner can thus guide the body 2 of the catheter between the two vocal chords 204. Once the distal end 14 is arranged in the trachea 203, the practitioner can administer the medicinal liquid in the form of microdroplets into the trachea of the subject through the distal orifice.

The administration of the medicinal liquid may be achieved by the activation of the pump connected to the reservoir of medicinal liquid. As explained above, the medicinal liquid may be administered in synchronization with the respiratory cycle of the subject 200, preferentially, the medicinal liquid is administered only during phases of inspiration of the subject 200.

The medicinal liquid is thus administered into the trachea 203 of the subject in the form of microdroplets 207, advantageously favoring its dispersion in the lungs of the subject while avoiding the “drowning” effect that the subject 200 could feel in the event of administration of a liquid and without use of a laryngoscope.

As already introduced, the medicinal liquid may be replaced by a medicinal powder delivered into the trachea in spray form.

### Other Applications

The catheter body according to the invention is preferentially designed to be inserted into a narrow channel such as the trachea of a premature newborn baby through its vocal chords. The diameter of such a trachea can reach 2 or 3 mm.

Those skilled in the art will understand that such a catheter designed to be inserted into the trachea through the vocal chords of the premature newborn subject will able to be used for similar applications such as genital and urinary applications. Notably, the diameter of the catheter body can enable its insertion through the urethra and/or the fallopian tubes. “Designed to be inserted into the trachea through the vocal chords of a premature newborn subject” is taken to mean that the catheter body is constructed, at the level of its dimensions, in such a way as to be able to be inserted into narrow vessels or channels of size similar to the trachea of a premature newborn baby such as the urethra, the cerebral vessels, or the fallopian tubes.

The catheter according to the invention also enables its use in ORL, digestive, endo peritoneal or endothoracic by laparoscopy applications. The catheter body is preferentially designed to be inserted into narrow channels less than 10 mm or 5 mm, very preferentially less than 3 mm.

1. A catheter for delivering a medicinal liquid or a powder in spray form into the trachea of a subject said catheter comprising a deformable catheter body designed to be inserted into the trachea through the vocal chords of the subject and comprising a spray device for expelling a liquid or a powder introduced into the catheter as a spray, said deformable catheter body comprising, further, a first lumen for the passage of an optic for generating an image of a zone situated in an extension of a distal end of said catheter and comprising a drive member for folding the deformable catheter body along a predetermined orientation.

2. The catheter according to claim 1, wherein the deformable catheter body comprises a second lumen defining a passage channel for supplying the spray device and a third lumen comprising the drive member.

3. The catheter according to claim 1, wherein said drive member makes it possible to drive an orientation of a distal part of the deformable catheter body with a proximal part of said deformable catheter body so as to control said orientation of the distal part in a predetermined direction.

4. The catheter according to claim 1, comprising a positioning piece intended to cooperate with the mouth of a subject, said positioning piece extending radially at least partially around the deformable catheter body to form a buccal support designed to limit a volume of air transiting outside the catheter between an inside and an outside of the mouth.

5. The catheter according to claim 4, wherein the positioning piece is translationally moveable on the deformable catheter body of the catheter.

6. The catheter according to claim 4, wherein the positioning piece comprises a guide to orientate the introduction of the catheter into the respiratory tracts of said subject.

7. The catheter according to claim 1, wherein the spray device comprises aerosolization means for expelling the medicinal liquid in aerosol form.

8. The catheter according to claim 1, wherein the drive member comprises:
a preformed tube extending into an orientation lumen of the deformable catheter body;
a removable rigid rod extending into a cavity of the preformed tube;
the preformed tube being designed in such a way that it comprises a preformed portion which, when the removable rigid rod is removed, takes a curved shape, inducing an angle between the parts of the tube on either side of said preformed portion in a predetermined plane.

9. The catheter according to claim 1, wherein said lumen for the passage of an optic is designed to receive an optic fiber extending along said lumen up to the distal end of the body of the deformable catheter catheter.

10. The catheter according to claim 9, wherein the optic fiber passage lumen comprises, at the distal end of the deformable catheter body, a transparent and leak tight wall for protecting the optic fiber passage lumen from contaminations of the external environment.

11. The catheter according to claim 1, wherein said optic fiber is also designed to light a zone situated in the distal extension of the catheter.

12. A medical system comprising a catheter according to claim 1, and comprising a command unit, said command unit comprising:
a reservoir for receiving a volume of medicinal liquid or a medicinal powder,
a device for commanding the administration respectively of the medicinal liquid or the medicinal powder for driving a high pressure pump.

13. The medical system according to claim 12, further comprising a display screen for displaying images of the optic.

14. The medical system according to claim 12, wherein the reservoir comprises means for controlling a temperature of the medicinal liquid or the medicinal powder in the reservoir.

15. An intubation system, comprising a catheter according to claim 1 and an intubation probe, said probe being designed to receive said catheter being capable of moving within said probe.

16. The catheter according to claim 1, wherein the subject is a premature newborn baby.
